(19) United States
(12) Patent Application Publication
(10) Pub. No.: US 2024/0210075 A1
(43) Pub. Date: Jun. 27, 2024

(54) SYRINGE CHILLER

(71) Applicant: PRP TECHNOLOGIES INC, Calgary (CA)

(72) Inventors: MARCIN KOWALEWSKI, Calgary (CA); Ryszard KOWALEWSKI, Calgary (CA)

(73) Assignee: PRP TECHNOLOGIES INC., Calgary, AB (CA)

(21) Appl. No.: 18/038,887

(22) PCT Filed: Jun. 15, 2021
(86) PCT No.: PCT/US21/37446

§ 371 Date: May 25, 2023

(51) Int. Cl. F25B 21/02; A61M 5/44

(52) U.S. Cl. CPC F25B 21/02; A61M 5/44; F25B 2321/0212; F25B 2321/0251; F25B 2700/2104

(57) ABSTRACT

A device for rapidly chilling a syringe, comprising: a thermal battery formed of a heat conductive material and including at least one chilling cavity defined therein; a thermoelectric cooler functioning as a heat pump, having a cold side placed in abutment with the thermal battery and having a hot side in abutment with an optional heatsink; a temperature sensor placed in abutment with the thermal battery; a temperature regulation module receiving a signal indicative of temperature from the temperature sensor, said temperature regulation module driving the thermoelectric cooler to achieve a pre-set temperature set-point; wherein the thermal battery reduces the temperature of the specimen to between 3 degrees C. and 20 degrees C. within 1-5 minutes.

### FIELD OF THE INVENTION

The present invention relates to a specialized cooling device for rapid chilling of a syringe. The device further relates to a device for rapid chilling of a syringe which may or may not contain a specimen. Still further, the device may be used for rapid chilling of blood and blood-derived fluids contained in syringes, specimen tubes or the like.

### BACKGROUND

There exists a long-felt but unmet need for a compact, easy-to-use device for chilling syringes. In the field of aesthetic medicine, physicians work to extract platelet rich plasma from whole blood for use in medical procedures. Blood begins to coagulate within thirty seconds after withdrawal from a human body. Anticoagulants are routinely used to slow down the coagulation process. When an anticoagulant is used in platelet rich plasma (PRP) preparation, a neutralizing agent is often used to reverse the effects of anticoagulation, prior to PRP administration. However, any exogenous additives may result in clinical side effects, cause platelet disfunction and raise the price of PRP. Low temperature and hypothermic conditions are known to slow the coagulation process. However, conventional refrigeration devices are big, bulky, expensive, and ill-suited to use in a clinical setting. What is needed is a device for rapidly cooling a syringe and accurately maintaining the temperature within a well-defined temperature range such that the contents of the syringe do not degrade and also do not freeze.

### SUMMARY OF THE INVENTION

Example 1: A device for rapidly chilling a syringe, comprising: a thermal battery formed of a heat conductive material and including at least one chilling cavity defined therein; a thermoelectric cooler whose cold side is in abutment with the thermal battery; a temperature sensor placed in abutment with the thermal battery; a temperature regulation module receiving a signal indicative of temperature from the temperature sensor, said temperature regulation module driving the thermoelectric cooler to achieve a pre-set temperature set-point; wherein the thermal battery reduces the temperature of the specimen to between 3 degrees C. and 20 degrees C. within 1-5 minutes.

Example 2: The device of Example 1, wherein the thermal battery reduces the temperature of the specimen to between 3 and 12 degrees Celsius within 1-5 minutes.

Example 3: The device of Example 1, wherein the thermal battery reduces the temperature of the specimen to between 3 and 8 degrees Celsius within 1-5 minutes.

Example 4: The device of Example 1, wherein the temperature regulation module includes an interface for setting the temperature set-point.

Example 5: The device of Example 4, wherein the interface includes a dial.

Example 6: The device of Example 1, wherein the temperature regulation module includes an indicator light which is illuminated when the temperature set-point is reached.

Example 7: The device of Example 1, wherein the temperature regulation module includes a first indicator light which is illuminated when the temperature set-point is reached and a second indicator light which is illuminated prior to achieving the temperature set-point.

Example 8: The device of Example 1, comprising a heatsink in abutment with the hot side of the thermoelectric cooler.

Example 9: The device of Example 8, comprising a fan proximate the heatsink.

Example 10: The device of Example 8, comprising a fan adjacent the heatsink.

Example 11: The device of claim 1, wherein driving the temperature regulation module comprises adjusting the voltage delivered to the thermoelectric cooler.

Example 12: The device of Example 1, wherein the thermal battery includes a liquid-tight housing including one or more portions formed of a heat conductive material, the liquid-tight housing containing a cooling fluid, the liquid-tight housing further containing at least one elongate tube having a chilling cavity defined therein, the at least one elongate tube at least partially immersed within the cooling fluid.

Example 13: The device of Example 12, wherein the at least one elongate tube is mounted to the liquid-tight housing.

Example 14: The device of Example 1 further comprising a stopper housed within the chilling cavity, the stopper having one of a lumen or a recess defined therein.

Example 15: A method for chilling a specimen of blood or blood-derived fluid within a syringe, using a device according to Example 1, comprising:
placing the syringe containing the specimen inside the chilling cavity; and
waiting for the specimen to reach between 3 and 20 degrees Celsius.

Example 16: The method of Example 15, wherein the specimen of blood or blood-derived fluid within the container does not include an anticoagulant.

### BRIEF DESCRIPTION OF THE DRAWINGS

FIGS. 1A-1B are block diagrams of exemplary syringe chillers;

FIGS. 2A-2D are enlarged views of a stopper within a chilling cavity;

FIG. 3 is a cross-sectional representation of the syringe chiller of FIGS. 1A-1B with a stopper of FIGS. 2A-2B;

FIGS. 4-5 depict the syringe chiller of FIG. 3 with a syringe or a specimen tube.

### DETAILED DESCRIPTION

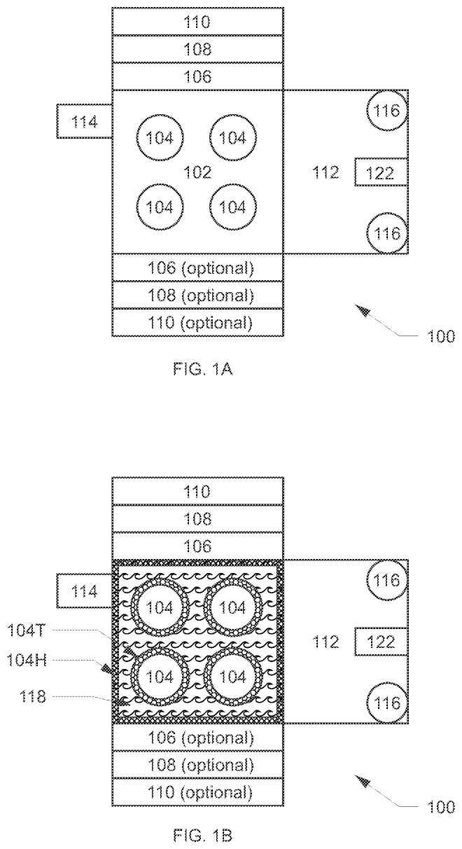

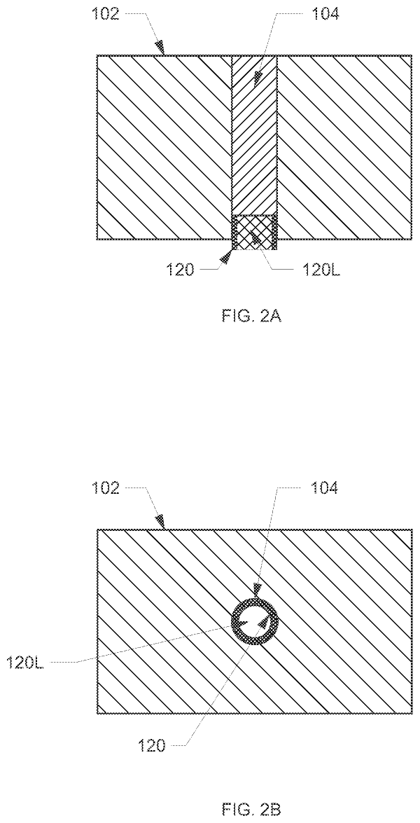

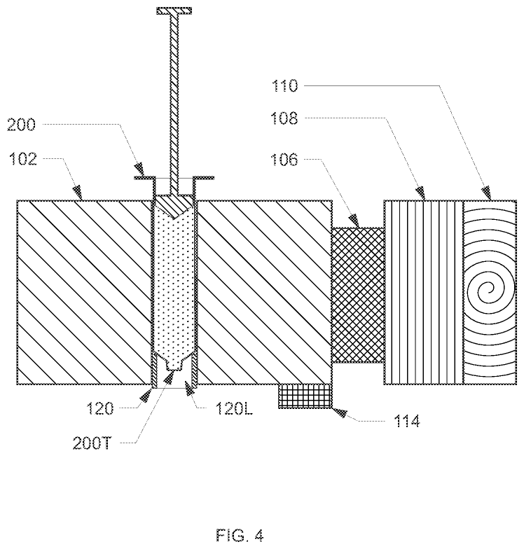

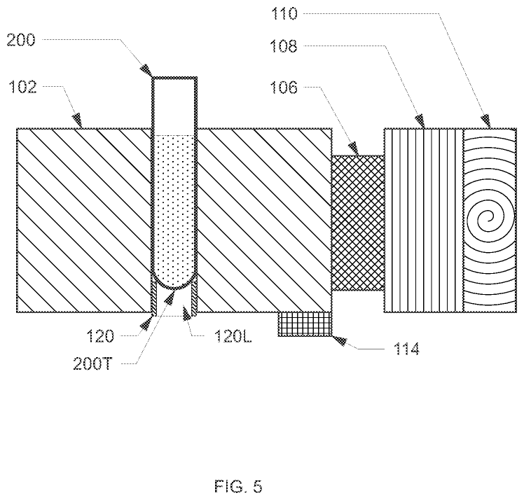

An example syringe chiller 100 is shown in FIG. 1A. The chiller 100 includes a thermal battery 102 having at least one chilling cavity 104 defined therein. The thermal battery is formed from a heat conducting material such as metal. The thermal battery 102 may be formed as a unitary piece of metal. In the example shown in FIG. 1A the thermal battery 102 is a block of aluminum. The thermal battery may be cast or machined. According to one example, the at least one chilling cavity 104 is machined into the block of aluminum. Throughout the present disclosure it should be understood that the term syringe 200 includes a specimen tube, container or vial and is not limited to a syringe. Thus, the syringe cavity means a cavity which can accommodate a syringe (FIG. 4), a specimen tube (FIG. 5) or other similarly shaped object. It is desirable for the diameter of the chilling cavity to closely approximate the diameter of the barrel of the syringe such that the walls of the chilling cavity are placed in intimate contact with the walls of the syringe barrel. Close approximation of the syringe barrel with the walls of the cavity will maximize the efficiency of heat exchange.

According to one example, the syringe chiller 100 is constructed to avoid contamination of the syringe. For example, the chilling cavity 104 isolates the syringe from contaminants or the like. The walls defining the chilling cavity 104 may contact the barrel of the syringe 200. In some examples, the distal tip 200T of the syringe preferably does not contact the chilling cavity wall or any part of the thermal battery 102 or any part of the entire device 100. The walls of the chilling cavity 104 may be anodized to prevent contamination of the syringe barrel by contact with the microparticles of the material comprising the chilling cavity wall.

According to one example the chilling cavity tapers 104 from wide to narrow to mirror the shape of the syringe.

According to one example a distal portion of the chilling cavity 104 is tapered.

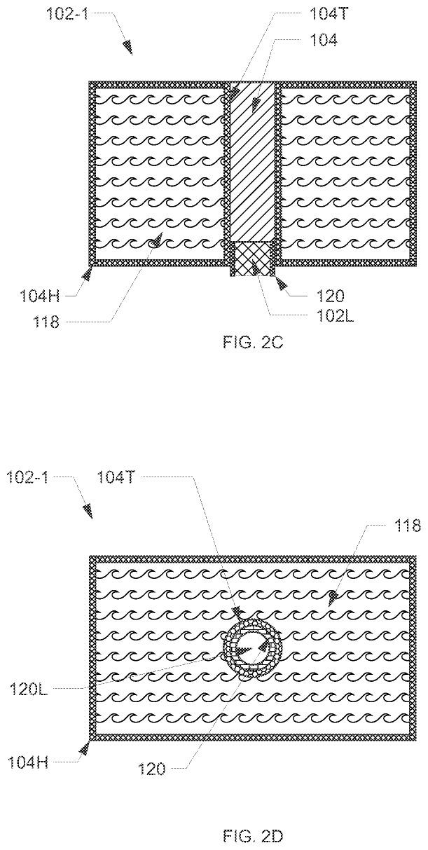

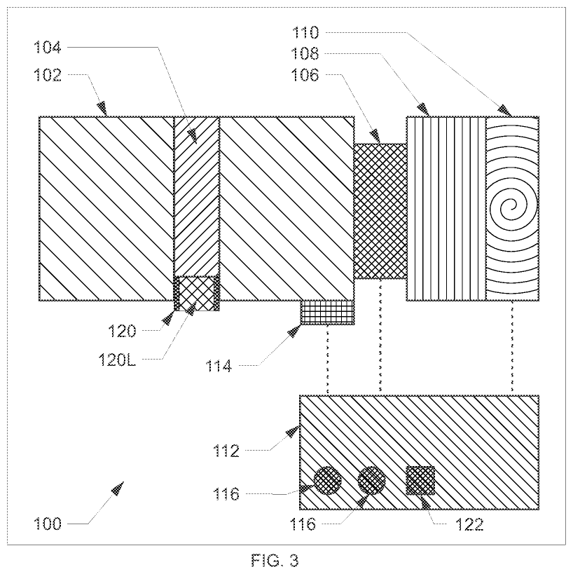

As shown in FIGS. 2A-5 the syringe chiller 100 may include an optional stopper 120 housed within the chilling cavity 104. The stopper 120 may include a lumen 120L defined therethrough sized to accommodate a distal end 200T of the syringe 200. The syringe stopper 120 is sized to fit within the chilling cavity 104. The stopper lumen 120L is sized to receive only the tapered tip 200T of a conventional syringe. See, FIGS. 4-5. The purpose of the stopper 120 is to ensure that the distal tip 200T of the syringe 200 is spaced apart from and does not contact the bottom of the thermal battery 102. The stopper 120 also ensures that a flangeless syringe does not fall through the chilling cavity 104. It acts as a stopper to position the syringe 200 within the chilling cavity 104 such that the fluid within the syringe is positioned within the depth of the cavity 104 to maximize the heat transfer efficiency. The lumen 120L of the stopper 120 accommodates the tip 200T of the syringe 200 in a way such that the tip does not contact any part of the syringe chiller 100.

According to another example, the thermal battery 102-1 (FIGS. 1B, 2C-2D) replaces the solid block of heat conductive material comprising thermal battery 102 in FIG. 1A. In this example, at least one syringe tube 104T is supported within a fluid-tight housing 104H containing a cooling fluid 118. The cooling fluid 118 surrounds the at least one syringe tube 104T and rapidly conducts heat away from the at least one syringe tube. The syringe tube 104T and at least a portion of the housing 104H may be formed of a heat conductive material such as metal (aluminum, copper, steel, or the like). The syringe tube 104T is a thermal conductor which transfers heat from the syringe to the cooling fluid 118. The syringe tube 104T at least partially houses the syringe 200 thereby creating the chilling cavity. The syringe tube 104T isolates the syringe 200 from direct contact with the cooling fluid 118. The cooling fluid 118 may be a mixture of water and alcohol or the like. In some examples, the cooling fluid 118 is a gel.

The thermal battery 102, 102-1 may be used without a cover thereby enabling a clinician to directly place or remove a syringe to/from the chilling cavity 104 without the need to open a cover.

The syringe chiller 100 further includes at least one thermoelectric cooler (TEC) 106 which may also be known as a Peltier module or a Peltier cooler. The TEC is a solid-state device that functions like a heat pump. In the examples depicted in FIGS. 1A-1B, the chiller includes two TEC's 106; however, the device could be equipped with a single TEC 106 or additional TEC's 106 as desired. Peltier modules or thermoelectric cooling modules are commercially available from numerous suppliers including Theremotekusa, Laird Thermal Systems Inc, Wakefield-Vette and MikroElectronika. The cooling surface, which is also known as the “cold side” of the TEC 106 is placed in abutment with the thermal battery 102, 102-1 to transfer heat from the battery 102 to the TEC 106. The TEC 106 is a flat component and has two distinct or polarized sides: a cold side and a hot side. When energized, the TEC 106 pumps heat from the cold side to the hot side. In this way, the TEC is “charging” the thermal battery, until it reaches the temperature set-point. When a warm syringe is placed into the chilling cavity 104, the syringe 200 gives off heat which becomes absorbed by the battery 102, 102-1 and “pumped” by the TEC 106 into the heatsink 108. The heatsink 108 then dissipates the heat to the surroundings. The attached fan 110 increases the air flow through the heatsink(s) 108. Increased airflow increases the heat transfer rate from heatsink 108 to air, and therefore minimizes the temperature rise on the heatsink 108, increasing the chilling efficiency even further.

To increase the thermal efficiency of the syringe chiller, the thermal battery may be insulated to minimize heat exchange with the ambient environment. The hot side of the TEC 106 is intentionally connected to the heatsink 108 or exposed to the ambient environment to allow the heat to dissipate into the ambient environment.

The TEC 106 has many advantages over conventional refrigeration technology which uses refrigerants. In particular, TEC 106 enables a compact form factor which is suitable for clinical uses. The TEC 106 is a solid-state device, which does not require recharging of fluids, and which does not require compression and decompression of fluids as a basis for its operation to transfer heat away from the thermal battery.

In some examples, a heatsink 108 may be placed in abutment with the TEC 106 to assist in dissipating heat from the TEC 106. Any known heat sink design may be used. The heatsink 108 is an optional but useful feature which increases the efficiency and performance of the syringe chiller 100 by providing a larger surface area for heat dissipation. In the example shown in FIG. 1A, 1B, the heatsink 108 is formed of a heat conducting material such as aluminum and includes a plurality of fins separated by air gaps. The heatsink 108 transfers heat from the TEC 106 to the environment via fins. In the example depicted in FIGS. 1A and 1B, the chiller 100 includes two heatsinks 108; however, the device could be implemented with a single heatsink 108 or additional heatsinks 108 as desired.

In some examples, the syringe chiller 100 may further be provided with one or more electric fans 110 to facilitate the dissipation of heat. The syringe chiller 100 may utilize one or more electric fans 110 as desired. The syringe chiller 100 may utilize both an electric fan 110 and a heatsink 108 as shown in FIGS. 1A-1B or may include a fan 110 only without the need for a heatsink 108. Again, the example shown in FIGS. 1A-1B includes two fans 110 and two heatsinks 108 however a single fan 110 and a single heatsink 108 may be used. The fan 110 increases airflow across the heat dissipating fins of the heatsink 108 and increases the efficiency of heat dissipation. Analogy to the wind chill effect: Normally the human body gives off little heat in the absence of air flow. However, given strong enough air flow (wind), a 24 degree C. ambient temperature may induce hypothermia.

In some examples, the TEC 106 includes a temperature regulation module 112 (FIG. 3) such that the desired temperature may be selected directly on the TEC 106. However, in other examples, a temperature regulation module 112 is provided which is distinct from the TEC 106. The temperature regulation module 112 is basically a controller or thermostat. The temperature regulation module 112 may include an interface or a dial for selecting a target temperature—a temperature set-point. The temperature regulation module 112 receives a signal from a temperature sensor 114 such as a thermocouple, thermistor or the like. The temperature sensor 114 may be mounted on the thermal battery 102, 102-1. The temperature regulation module 112 includes logic which compares the temperature set-point with the signal received from the temperature sensor 114 and adjusts the voltage delivered to the TEC 106 as necessary to achieve the temperature set-point.

In some examples, the temperature regulation module 112 includes an indicator light 116 which is illuminated when the temperature set-point is reached. In some examples, the indicator light includes a first indicator light 116 which is illuminated when the temperature set-point is reached and a second indicator light 116 which is illuminated prior to achieving the temperature set-point. In some examples, the temperature regulation module 112 includes a speaker 122 which provides an audible signal when the temperature set-point is reached.

The present invention is ideally suited for rapidly chilling a syringe containing a blood specimen. In particular, a blood specimen without an anticoagulant. Chilling the blood specimen slows coagulation—even in the absence of an anticoagulant. This is advantageous in situations in which the use of an anticoagulant is contraindicated, e.g., medical procedures utilizing platelet rich plasma.

It is desirable to rapidly chill the blood specimen without freezing the blood and without damaging the platelets. If the blood specimen is chilled too quickly the platelets may burst, and if the blood specimen is chilled too slowly then the blood may coagulate. In the case of PRP preparation, if the blood is chilled too much (becomes too cold), on account of increased density the blood will be difficult to separate in the centrifuge and may require significantly longer centrifugation time. On the other hand, if the blood is not chilled enough (remains too warm), it may coagulate while still in the centrifuge, or the obtained blood derivatives may coagulate prior to injection, rendering the fluid non-injectable. The freezing point of blood is approximately −2 degrees Celsius. It is desirable to reduce the temperature to between 3 C and 20 C within 1 to 5 minutes. It is further desirable to reduce the temperature to between 3 C and 12 C within 1 to 5 minutes. Further still it is desirable to reduce the temperature to between 3 C and 8 C within 1 to 5 minutes. In some situations, the goal is to slow coagulation while still facilitating centrifugation to separate the blood specimen into its constituent parts.

While the present disclosure has been described with reference to various embodiments, these embodiments are illustrative, and the scope of the disclosure is not limited to them. Many variations, modifications, additions, and improvements are possible. More generally, embodiments in accordance with the present disclosure have been described in the context of particular embodiments. Functionality may be separated or combined in procedures differently in various embodiments of the disclosure or described with different terminology. These and other variations, modifications, additions, and improvements may fall within the scope of the disclosure as defined in the claims that follow.

1. A device for rapidly chilling a syringe containing a specimen, comprising:
a thermal battery formed of a heat conductive material and including at least one chilling cavity defined therein;
a thermoelectric cooler having a cold side placed in abutment with the thermal battery;
a temperature sensor placed in abutment with the thermal battery; and
a temperature regulation module receiving a signal indicative of temperature from the temperature sensor, said temperature regulation module driving the thermoelectric cooler to achieve a pre-set temperature set-point; wherein the thermal battery reduces the temperature of the specimen to between 3 degrees C. and 20 degrees C. within 1-5 minutes.

2. The device of claim 1, wherein the thermal battery reduces the temperature of the specimen to between 3 and 12 degrees Celsius within 1-5 minutes.

3. The device of claim 1, wherein the thermal battery reduces the temperature of the specimen to between 3 and 8 degrees Celsius within 1-5 minutes.

4. The device of claim 1, wherein the temperature regulation module includes an interface for setting the temperature set-point.

5. The device of claim 4, wherein the interface includes a dial.

6. The device of claim 1, wherein the temperature regulation module includes an indicator light which is illuminated when the temperature set-point is reached.

7. The device of claim 6, wherein the indicator light includes a first indicator light which is illuminated when the temperature set-point is reached and a second indicator light which is illuminated prior to achieving the temperature set-point.

8. The device of claim 1, comprising a heatsink in abutment with the hot side of the thermoelectric cooler.

9. The device of claim 8, comprising a fan proximate the heatsink.

10. The device of claim 8, comprising a fan adjacent the heatsink.

11. The device of claim 1, wherein driving the temperature regulation module comprises adjusting the voltage delivered to the thermoelectric cooler.

12. The device of claim 1, wherein the thermal battery includes a liquid-tight housing including one or more portions formed of a heat conductive material, the liquid-tight housing containing a cooling fluid, the liquid-tight housing further containing at least one elongate tube having a chilling cavity defined therein, the at least one elongate tube at least partially immersed within the cooling fluid.

13. The device of claim 12, wherein the at least one elongate tube is mounted to the liquid-tight housing.

14. The device of claim 12 further comprises: a lid to close the liquid-tight housing.

15. The device of claim 1, further comprising a stopper housed within the chilling cavity, the stopper having one of a lumen or a recess defined therein.

16. A method for chilling a specimen of blood within a syringe, comprising:
providing a chilling device, including:
a thermal battery formed of a heat conductive material and including at least one chilling cavity defined therein;
a thermoelectric cooler having a cold side placed in abutment with the thermal battery;
a temperature sensor placed in abutment with the thermal battery; and
a temperature regulation module receiving a signal indicative of temperature from the temperature sensor, said temperature regulation module driving the thermoelectric cooler to achieve a pre-set temperature set-point; wherein the thermal battery reduces the temperature of the specimen to between 3 degrees C. and 20 degrees C. within 1-5 minutes;
placing the syringe containing the specimen inside the chilling cavity; and
waiting for the specimen to reach between 3 and 20 degrees Celsius.

17. The method of claim 16, wherein the specimen of blood within the container does not include an anticoagulant.
